(19) United States
(12) Patent Application Publication
(10) Pub. No.: US 2006/0106271 A1
(43) Pub. Date: May 18, 2006

(54) Process for the recovery of an ethylene and propylene containing stream from a cracked gas resulting from hydrocarbon cracking

(75) Inventors: Theodorus Petru Everardus Antonius Hubbers, Heerlen (GB); Gerhard Schembecker, Dortmund (DE); Axel Gottschalk, Hattingen (DE); Hendrik Derk Leunk, Vrijheidslaan (DE)

Correspondence Address: MAYER, BROWN, ROWE & MAW LLP, 1909 K STREET, N.W., WASHINGTON, DC, 20006, US

(73) Assignee: MAYER BROWN ROWE 7 MAW LLP

(21) Appl. No.: 10/520,179

(22) PCT Filed: Jun. 27, 2003
(86) PCT No.: PCT/NL03/00474

§ 371 Date: Nov. 1, 2005

(30) Foreign Application Priority Data
Jul. 5, 2002 (EP) 02077/666.2

(51) Int. Cl. C07C 4/02; C07C 7/00

(52) U.S. Cl. 585/650; 585/648; 585/500

(57) ABSTRACT

Process for the recovery of an ethylene and propylene containing stream from a cracked gas resulting from cracking a hydrocarbon stream, wherein the cracked gas is treated in an absorptive demethanizer with a C₄/C₅ solvent at a temperature between −10° C. and −40° C. to free the cracked gas from methane and hydrogen gas, whereafter the remaining stream is treated by distillation in a distillation unit to obtain a C₄/C₅ containing stream and the ethylene and propylene containing stream; whereafter the C₄/C₅ stream is treated with a hydrogen containing stream in a hydrogenation unit, whereafter a part of the hydrogenated C₄/C₅ stream is cooled to a temperature between −10° C. and −40° C. and recycled to the absorptive demethanizer and a part of the hydrogenated C₄/C₅ stream is separated.

The invention relates to a process for the recovery of an ethylene and propylene containing stream from a cracked gas resulting from cracking a hydrocarbon stream.

The hydrocarbon cracking to obtain ethylene and propylene is disclosed in for example Ullmann's Encyclopedia of Industrial Chemistry, Vol. A 10, 1996, p. 45-93. Besides the ethylene and the propylene also other products for example cracked fuel oil, hydrogen, methane, C₄-material and pyrolysis gasoline result from the hydrocarbon cracking. Some of these products have commercial value but actually a lot of these products are only undesired coproducts. Generally, the recovery of ethylene and propylene with a good yield is the main aim for hydrocarbon cracking.

It is the object of the present invention to recover an ethylene and propylene containing stream from a hydrocarbon cracker whereas the undesired coproducts are not obtained in the recovery section as separate products.

The invention is characterized in that the cracked gas is treated in an absorptive demethanizer with a C₄/C₅ solvent at a temperature between −10° C. and −40° C. to free the cracked gas from methane and hydrogen gas, whereafter the remaining stream is treated in a distillation unit to obtain a C₄/C₅ containing stream and the ethylene and propylene containing stream; whereafter the C₄/C₅ stream is treated with a hydrogen containing stream in a hydrogenation unit, whereafter a part of the hydrogenated C₄/C₅ stream is cooled to a temperature between −10° C. and −40° C. and recycled to the absorptive demethanizer and a part of the hydrogenated C₄/C₅ stream is separated and may, for instance, be recycled to the hydrocarbon cracker.

Hydrogen, C₄ and Cs material are used within the process according to the invention and consequently they are not recovered as separate products.

Another advantage of the present process is the improved yield of ethylene and propylene.

Preferably, the propylene/ethylene ratio (P/E ratio) is higher than 0.55.

In the process according to the invention an absorptive demethanizer is used and as a result thereof no deep cooling is necessary and no expensive material is necessary for the demethanizer column.

The cracked gas resulting from hydrocarbon cracking is obtained by cooling and compressing the gas resulting from cracking. In the compression section the cracked gas is compressed in several stages and the hydrocarbons containing 6 or more carbon atoms (C₆⁺) are removed from the cracked gas. Acid gases may, for instance, be removed in a caustic wash tower and the heavy ends may be removed from the cracked gas by distillation. It is also possible to hydrogenate the cracked gas in a front-end hydrogenation unit before introduction in the absorptive demethanizer to remove acetylenes and propadiene from the cracked gas.

The hydrogenation is performed by contacting the cracked gas with a hydrogenation catalyst. Hydrogenation is performed at a pressure between 1.5 and 5.0 MPa and a temperature between 25° C. and 100° C. Generally, a hydrogenation catalyst contains a metal compound comprising Ni, Pd, Pt and/or Co.

Preferably, the catalyst is a Pd-containing catalyst.

Preferably, the hydrogenation catalyst is a supported catalyst. Examples of suitable carrier materials are silica, α-, θ and γ-alumina, zeolites, carbon and oxidic carriers, such as for instance magnesium oxide, titanium oxide and/or zirconium oxide. By preference, the carrier material is θ-and γ-alumina, silica or carbon. Most preferably θ-or γ-alumina are used as carrier material because this is an inert carrier material with a large total surface area and a good poor volume distribution. Generally, the metal content of the catalyst ranges between 10 and 25 wt %. Supported catalysts are commercially available with for instance 10, 15 and 20 wt % of metal.

Additionally the catalyst may also contain minor amounts of compounds which enhance the activity and selectivity of the catalyst. Examples of such compounds include: chromium, gold, rhodium and/or ruthenium. The catalyst may also be modified with a sulphur-containing compound.

The C₄/C₅ solvent is present within the loop comprising the absorptive demethanizer, a distillation unit and a hydrogenation unit. The C₄/C₅ stream comprises all compounds with 4 or 5 carbon atoms out of the cracked gas. An amount less than 15 wt % of hydrocarbon compounds with 6 or more carbon atoms is allowed to be present in the C₄/C₅ stream.

The cracked gas is first treated in an absorptive demethanizer to free the cracked gas from methane and hydrogen by using a C₄/C₅ solvent and a cooling medium simultaneously. It dissolves the hydrocarbons out of the cracked gas, except the hydrogen gas and methane, which are removed from the absorptive demethanizer. The temperature in the absorptive demethanizer is between −10° C. and −40° C. and the pressure is between 0.1 and 5.0 MPa.

The remaining stream, being the C₄/C₅ stream combined with the hydrocarbons having 2-5 carbon atoms out of the cracked gas, is treated by distillation at a pressure between 0.5 and 2.0 MPa and at a temperature between −40° C. and 150° C. An ethylene and propylene containing stream is obtained and the C₄/C₅ stream is thereafter fed to a hydrogenation unit where it is substantially or partly hydrogenated. A part of the C₄/C₅ stream is separated from the loop after hydrogenation. The remaining stream is cooled and recycled as a solvent to the absorptive demethanizer.

It is possible to apply more than one hydrogenation unit. The hydrogenation may be performed with hydrogen gas from the absorptive demethanizer or with hydrogen gas from another source. Preferably, the hydrogen gas resulting from the absorptive demethanizer is used for the hydrogenation. This hydrogen gas may be used as a hydrogen mixture with methane as it is resulting from the absorptive demethanizer or it can first be separated from the methane gas.

The reactor for the hydrogenation may be a packed-bed or a trickle-bed reactor. The flow within the reactor is a cocurrent or a countercurrent flow. The hydrogenation temperature generally ranges between 25° C. and 100° C.

The C₄/C₅ stream may be substantially or partly hydrogenated to remove the unsaturated compounds. In case the C₄/C₅ stream is substantially hydrogenated the C₄/C₅ stream comprises at most 15 wt % of unsaturated compounds. In case the C₄/C₅ stream is partly hydrogenated the amount of unsaturated compounds in the C₄/C₅ stream is between 50 and 90 wt % and preferably between 70 and 90 wt %.

When the C₄/C₅ stream is substantially hydrogenated a part of it is separated from the loop and may, for instance, be recycled to the hydrocarbon cracker.

When the C₄/C₅ stream is partly hydrogenated a part of it is separated from the loop, it is preferably treated by catalytic cracking. The aim of catalytic cracking is to recover ethylene and propylene out of the unsaturated C₄/C₅ stream. By the catalytic cracking of the C₄/C₅ stream not only the yield of ethylene and propylene obtained with the process according to the invention is higher, but also the propylene/ethylene ratio (P/E ratio) is improved. By the catalytic cracking of the unsaturated C₄/C₅ stream a P/E ratio higher than 0.70, preferably higher than 0.75, may be obtained.

The catalytic cracking may be performed in one or more reactors. The temperature during catalytic cracking is between 400° C. and 800° C. and the pressure is between 0.05 and 0.5 MPa. All known processes for catalytic cracking can be used.

The ethylene and propylene containing stream obtained after distillation of the C₄/C₅ stream combined with the hydrocarbons with 2-5 carbon atoms out of the cracked gas is led to a chemical absorption unit. In the chemical absorption unit ethylene and propylene are chemically absorbed from the ethylene and propylene containing stream, in a solvent containing a compound derived from a metal of group 10 or 11 of the Periodic Table of the Elements, followed by recovery of ethylene and propylene from said solvent by heating and/or by reducing the pressure. The ethylene and propylene-containing stream is substantially free of hydrogen, acetylenes and dienes.

The Periodic Table of the Elements is disclosed on the inside of the cover of the Handbook of Chemistry and Physics, 70^{th} edition, 1989/1990.

The ethylene and propylene containing stream mainly comprises ethylene, ethane, propylene and propane. Ethylene and propylene are separated from ethane and propane by subjecting the ethylene and propylene containing stream to a chemical absorption process. The chemical absorption process is performed by contacting the ethylene and propylene containing stream and a solvent containing a compound derived from a metal of group 10 or 11 of the Periodic Table of the Elements.

Preferably, the metal compound comprises Cu or Ag. Examples of suitable Ag-containing compounds are silver acetate, silver nitrate and/or silver tetrafluoroborate. Examples of suitable Cu-containing compounds are cuprous nitrate, cuprous sulphate, cuprous tetrafluoroborate, cuprous tetrachloroaluminate and/or cuprous diketonate.

Suitable solvents used in the chemical absorption process are water or aromatic and olefinic solvents. Examples of aromatic and olefinic solvents are alfa-methylstyrene, benzene and toluene. Generally, Ag-containing compounds are used in combination with water as the solvent whereas Cu-containing compounds may be used with water as the solvent and also in the presence of a stabilizer such as, for instance ammonia, pyridine or an alkanolamine. Cu-containing compounds are usually dissolved in aromatic or olefenic solvents.

Most preferably, the solvent is water and the metal compound is AgNO₃. The concentration of the compound in the solvent may be between 2 and 15 Mol/l.

The ethylene and propylene containing stream is contacted with the solvent for chemical absorption at a temperature between 0° C. and 50° C. and a pressure of 0.5 to 3.0 MPa.

Ethane and propane may be recycled to the hydrocarbon cracker.

Ethylene and propylene which are absorbed are recovered from the solvent by heating and/or by reducing the pressure. It is also possible to heat the solvent first, where after the pressure is reduced or vice versa.

Preferably, the temperature and the pressure at which ethylene and propylene are released from the solvent is between 80° C. and 150° C. and between 0.1 and 1.0 MPa. Thereafter this product stream is separated in an ethylene stream and a propylene stream by distillation at a pressure between 0.1 and 3.0 MPa and a temperature between −60° C. and 100° C.

The invention is also directed to a recovery section of a hydrocarbon cracker and the invention is also directed to a method to modify an existing hydrocarbon cracker by providing it with a recovery section according to the invention.

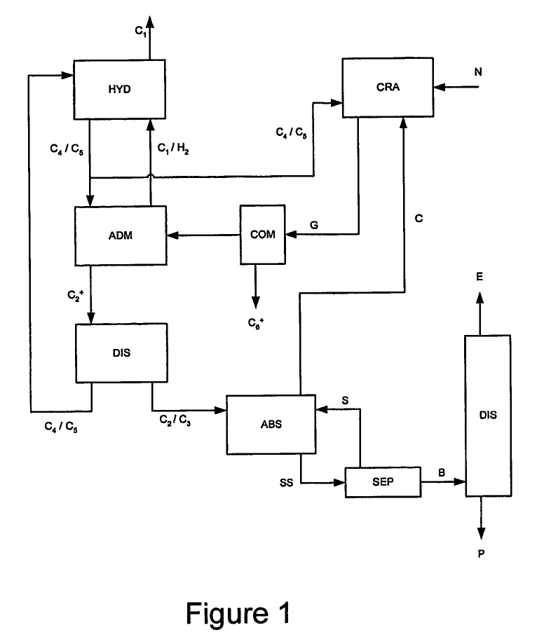

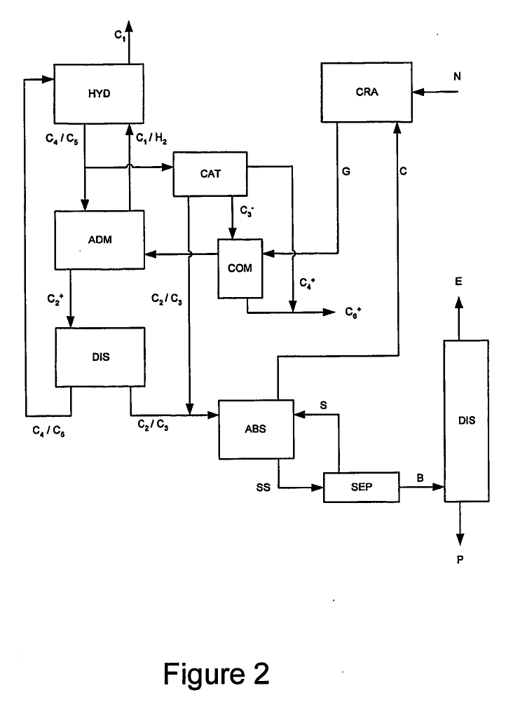

The invention will be further explained with respect to FIG. 1 and FIG. 2 without being limited thereto.

### EXAMPLE I

FIG. 1 shows the recovery section of a hydrocarbon cracker. A naphtha feedstock (N) (1000 t/h) was cracked in a hydrocarbon cracker (CRA). In the compression section (COM) the cracked gas was compressed in several stages and the hydrocarbons containing 6 or more carbon atoms (C₆⁺) were removed from the cracked gas.

The cracked gas was thereafter led to an absorptive demethanizer (ADM) where it was treated with a C₄/C₅ solvent (C₄/C₅) at a temperature of −30° C. The composition of the C₄/C₅ solvent was 50 wt % n-butane, 15 wt % i-butane, 10 wt % n-pentane, 5 wt % i-pentane, 10 wt % cyclopentane, 2 wt % other C₄ and C₅ compounds and 8 wt % C₆ compounds.

In the ADM hydrogen gas and methane (C₁/H₂) were separated from the cracked gas and the resulting stream (C₂⁺) was led to a distillation unit (DIS) where the stream was treated at 0.1 MPa; top temperature −19° C. and bottom temperature 86° C.; in an ethylene and propylene containing stream (C₂/C₃) and a C₄/C₅ stream (C₄/C₅).

The C₂⁺ stream comprised 23 wt % ethylene, 13 wt % propylene, 27 wt % n-butane, 8 wt % i-butane, 6 wt % n-pentane, 3 wt % I-pentane, 6 wt % cyclopentane and 12 wt % other compounds.

The C₂/C₃ stream comprised 2 wt % methane, 9 wt % ethane, 57 wt % ethylene, 1 wt % propane and 31 wt % propylene.

The C₄/C₅ stream comprised 45 wt % n-butane, 14 wt % i-butane, 11 wt % n-pentane, 4 wt % I-pentane, 9 wt % cyclopentane, 8 wt % other C₄ compounds and 9 wt % other C₅ compounds.

This C₄/C₅ stream was led to a hydrogenation unit. Hydrogenation is performed with C₁/H₂ coming from the ADM. After hydrogenation C, was separated about 20 wt % of C₄/C₅ was separated and recycled to the CRA. The other part of C₄ was cooled to −35° C. and introduced in the ADM.

The C₂/C₃ stream was led to a chemical absorption unit (ABS) and was contacted in this unit with a solvent (S) containing AgNO₃ at a temperature of 25° C. and a pressure of 1.0 MPa. The concentration of the AgNO₃ in the solvent was 6 mol/l. The solvent was water. Ethylene and propylene were absorbed in the solvent and ethane and propane (C) were removed from ABS. C was recycled to a hydrocarbon cracker. The solvent containing ethylene and propylene (SS) was led to a separation unit wherein the pressure was released to 0.6 MPa. Thereafter the temperature in the separation unit was raised to 100° C. to remove ethylene and propylene from it whereafter S was recycled to ABS. The combined ethylene and propylene stream (B) was led to a distillation unit to separate B in ethylene (E) and propylene (P). The pressure during distillation was 1.8 MPa. The top temperature was −32° C. and the bottom temperature was 44° C.

An amount of 387 t/h of E with a purity of 99.999 wt % was obtained and an amount of 215 t/h of P with a purity of 99.98 wt % was obtained.

### EXAMPLE II

In FIG. 2 the recovery section is the same as the recovery section according to FIG. 1. The cracking process and recovery process that was performed was based on the same cracked gas as the process according to Example I.

Naphtha was fed to the hydrocarbon cracker with 1000 t/h.

In FIG. 2 the hydrogenation unit (HYD) is so designed that not all unsaturated compounds in C₄/C₅ are hydrogenated and the part of C₄/C₅ that is separated is treated by catalytic cracking with a zeolite catalyst at a temperature of 560° C. and a pressure of 0.15 MPa. After catalytic cracking the C₄/C₅ stream was separated in 2 different streams by distillation. An extra ethylene and propylene containing stream was obtained which was, together with the C₂/C₃ stream resulting from DIS introduced in ABS.

Further a C₄⁺ stream, containing a lot of aromatics, was obtained that was separated together with the C₆⁺ stream and a stream containing hydrocarbons with 1 to 3 carbon atoms comprising some hydrogen. This stream (C₃⁻) was recycled to COM.

An amount of 359 t/h of E with a purity of 99.999 wt % and an amount of 274 t/h of P with a purity of 99.98 wt % was obtained.

1. A process for the recovery of an ethylene and propylene containing stream from a cracked gas resulting from cracking a hydrocarbon stream, wherein the cracked gas is treated in an absorptive demethanizer with a C₄/C₅ solvent at a temperature between −10° C. and −40° C. to free the cracked gas from methane and hydrogen gas, whereafter the remaining stream is treated by distillation in a distillation unit to obtain a C₄/C₅ containing stream and the ethylene and propylene containing stream; whereafter the C₄/C₅ stream is treated with a hydrogen containing stream in a hydrogenation unit, whereafter a part of the hydrogenated C₄/C₅ stream is cooled to a temperature between −10° C. and −40° C. and recycled to the absorptive demethanizer and a part of the hydrogenated C₄/C₅ stream is separated.

2. A process according to claim 1 wherein the C₄/C₅ stream is hydrogenated with the use of the hydrogen gas coming from the absorptive demethanizer.

3. A process according to claim 1, wherein the C₄/C₅ stream is substantially hydrogenated in the hydrogenation unit.

4. A process according to claim 1, wherein the C₄/C₅ stream is partly hydrogenated in the hydrogenation unit and part of the C₄/C₅ stream is separated after the hydrogenation unit and treated by catalytic cracking, whereafter an additional ethylene and propylene containing stream is obtained.

5. A process according to claim 1, wherein from the ethylene and propylene containing stream, being substantially free of hydrogen, acetylenes and dienes, ethylene and propylene are chemically absorbed in a solvent containing a compound derived from a metal of group 10 or 11 of the Periodic Table of the Elements, followed by recovery of ethylene and propylene from said solvent by heating and/or by reducing the pressure.

6. A process according to claim 1, wherein the propylene/ethylene ratio in the ethylene and propylene containing stream is higher than 0.55.

7. A process according to claim 4, wherein the propylene/ethylene ratio in the combined ethylene and propylene containing stream is higher than 0.70.

8. A recovery section of a hydrocarbon cracker comprising an absorptive demethanizer, a distillation unit and a hydrogenation unit wherein a process according to claim 1 is applied.

9. A recovery section according to claim 8, wherein the hydrogenation in the hydrogenation unit takes place with hydrogen gas from the absorptive demethanizer.

10. A method to modify an existing hydrocarbon cracker by providing it with a recovery section according to claim 8.

11. A process according to claim 2, wherein the C₄/C₅ stream is substantially hydrogenated in the hydrogenation unit.

12. A process according to claim 2, wherein the C₄/C₅ stream is partly hydrogenated in the hydrogenation unit and part of the C₄/C₅ stream is separated after the hydrogenation unit and treated by catalytic cracking, whereafter an additional ethylene and propylene containing stream is obtained.

13. A process according to claim 2, wherein from the ethylene and propylene containing stream, being substantially free of hydrogen, acetylenes and dienes, ethylene and propylene are chemically absorbed in a solvent containing a compound derived from a metal of group 10 or 11 of the Periodic Table of the Elements, followed by recovery of ethylene and propylene from said solvent by heating and/or by reducing the pressure.

14. A process according to claim 3, wherein from the ethylene and propylene containing stream, being substantially free of hydrogen, acetylenes and dienes, ethylene and propylene are chemically absorbed in a solvent containing a compound derived from a metal of group 10 or 11 of the Periodic Table of the Elements, followed by recovery of ethylene and propylene from said solvent by heating and/or by reducing the pressure.

15. A process according to claim 2, wherein the propylene/ethylene ratio in the ethylene and propylene containing stream is higher than 0.55.

16. A process according to claim 2, wherein the propylene/ethylene ratio in the combined ethylene and propylene containing stream is higher than 0.70.
